(19) United States
(12) Patent Application Publication
(10) Pub. No.: US 2018/0045981 A1
(43) Pub. Date: Feb. 15, 2018

(54) ELECTROCHROMIC DEVICE ADAPTED FOR HEATING TO PREVENT FOGGING

(71) Applicant: ABOMINABLE LABS, LLC, Lake Oswego, OR (US)

(72) Inventors: JACK C. CORNELIUS, Lake Oswego, OR (US); VINCENT O'MALLEY, Portland, OR (US)

(73) Assignee: ABOMINABLE LABS, LLC, Lake Oswego, OR (US)

(21) Appl. No.: 15/679,106

(22) Filed: Aug. 16, 2017

Related U.S. Application Data
(60) Provisional application No. 62/374,887, filed on Aug. 14, 2016.

(51) Int. Cl. G02C 11/08; A61F 9/02; G02F 1/15

(52) U.S. Cl. CPC G02C 11/08; G02F 1/1521; A61F 9/023; A61F 9/026; G02C 11/04

(57) ABSTRACT

Portable, light attenuating electrochromic device adapted for heating to prevent fogging and to enhance operability during colder weather comprising: opposed substrates defining an enclosed space for receiving a liquid crystal solution and having conducting layers, the first substrate having a heating element system thereon for controlled operation via a first voltage power supply circuit between an upper voltage limit and a lower voltage limit, the second substrate having a tint control system thereon for controlled operation via a second voltage power supply circuit at first and second state tint voltages outside the heating voltage range upper and lower voltage limits, for heating the device during cold-weather operation to prevent fogging of the device and for attenuating light through the device to account for varying ambient lighting conditions despite colder weather operating conditions.

### CROSS-REFERENCE TO RELATED APPLICATIONS

This patent application claims the benefit and priority of U.S. Provisional Patent Application Ser. No. 62/374,887, for ELECTROCHROMIC DEVICE ADAPTED FOR HEATING TO PREVENT FOGGING, filed 14 Aug. 2016.

### FIELD OF INVENTION

This invention relates generally to a cell with electronically attenuated light transmission, and more particularly to a cell with electronically attenuated light transmission capability that is also adapted for heating to prevent fogging, and in the case of use in cold weather temperatures, to improve performance of the cell when used in the cold.

### BACKGROUND OF THE INVENTION

There are prior electrochromic devices which make use of a liquid-crystal cell, or a cell with dichroic dyes and the like for electronically attenuating light transmission through lenses or displays. An example of one of these is described in U.S. Pat. No. 5,015,086 for Electronic Sunglasses, to Okaue et al. Another example of one of these is described in U.S. Pat. No. 6,239,778 for Variable Light Attenuating Dichroic Dye Guest-Host Device, to Palffy-Muhoray et al. These devices may be useful for allowing light transmission through the cell, as for example with eye-glasses, goggles, or viewing screens, to the degree that they provide controllable and very rapid attenuation of light transmission under optimal temperature conditions. Another important benefit of these devices is the degree to which they have been able to be used with glass or plastic substrate cells, and the degree to which they have been able to be designed to accommodate any color or tint. Further, these devices have provided a fail-safe device (biasing to more or less opacity depending on the application) when no electrical power is supplied. Thus, for example, in the case where unobscured vision is critical, such as with military goggles and the like, they have been biased to allow high light transmittance to allow vision through the cell when the power-source fails, thus preventing a vision screen (such as in eye-glasses or a visor), or a goggle, from going dark and preventing vision if the batteries fail. Alternatively, of course, this bias may have been in another direction so that the lens goes darker if the batteries fail, as might be desirable for example for a lens in a welding helmet.

It is often desirable to use sport goggles, tactical goggles, dive masks and other highly portable transparent eye-protecting shields, wearable virtual reality or augmented reality devices, or other devices having view displays, or vision screens, in environments involving conditions, which are conducive to fogging and may also be exposed to colder weather temperatures, which may also contribute to condensation build-up on the eye shield or display. With such devices, activities and environments, even momentary impairment of vision by fogging would be problematic. When the temperature of such an eye shield, vision screen or display has dropped below a dew-point temperature, i.e., the atmospheric temperature below which water droplets begin to condense and dew can form, fogging has occurred. And yet, because such devices have needed to be portable, and therefore typically have had limited size battery power systems, such systems have needed to use power highly efficiently in order to have enabled sufficient battery life to have allowed use of the device for extensive periods of time, on the order of at least 6 hours between charges, to have been useful under various weather conditions.

There have been various conductive apparatus devised for preventing condensation build-up on eye-shields and other displays. The purpose of these conductive apparatus has been to provide an eye shield that may be maintained free of condensation so that the user would be able to enjoy unobstructed vision during viewing activities. Prior goggles and wearable gaming devices with electronic systems have been primarily used in environments requiring a high degree of portability, that is, where a power source for powering the electronics for the device has been advantageously carried on a strap for the goggle or on the goggle itself as shown and described in U.S. Pat. No. 9,301,879 to McCulloch et al., for Goggle with Easily Interchangeable Lens that is Adaptable for Heating to Prevent Fogging.

As their name suggests, liquid-crystals exist in a state that is similar to both a liquid and a solid in the same material. Thus, in this state, their molecules tend to maintain their orientation, like the molecules in a solid, but also move around to different positions, like the molecules in a liquid, responsive to small electrical currents to which the crystals have been subjected.

Further, some of these devices, such as in particular goggles for use in snow sports, work or tactical activities, gaming virtual reality or augmented reality devices, or for use in hand-held GPS or radio devices, have often been used in weather conditions conducive not only to fogging of a lens or display, but have also often been used during very cold weather situations, for example below −20 degrees Celsius, where the more extreme cold has begun to diminish, or beyond which temperature has rendered completely ineffective, such devices. In the case of the Palffy-Muhoray device, for example, since the host material for the dichroic dye guest is liquid-crystal, these devices have suffered from some of the known vulnerability that liquid-crystal devices have had to cold weather operability generally. This is because the liquid-crystals are closer to a liquid state, than a solid state, the liquid-crystals being susceptible to reduced free flow in very cold temperatures. Accordingly, in such very cold weather operating conditions, liquid-crystal electronic light attenuating devices have been incapable of functioning optimally, because the orientation of the liquid-crystals and associated dyes have become frozen, or at least thickened, so as to have been less fluid and more limited in their ability to change orientation to decrease/increase light transmittance. Because the liquid-crystals need to be free flowing to change their orientation for the transmittance of light to be rapidly and freely varied responsive to voltage changes within the device, this freezing, or thickening, of the liquid-crystals has prevented a more rapid change in orientation of the crystals, and their associated dyes to vary opacity, and this has prevented proper, and especially rapid, functioning of the device.

Examples of fog-prone goggles intended for use during winter activities have included goggles for downhill skiing, cross-country skiing, snowboarding, snowmobiling, sledding, tubing, ice climbing, military issue goggles, and the like, and are widely known and widely utilized by sports enthusiasts and others whose duties or activities have required them to be outside in snowy and other inclement cold-weather conditions. Examples of fog-prone dive masks have included eye and nose masks independent of a breathing apparatus as well as full-face masks in which the breathing apparatus is integrated into the mask. Examples of fog-prone eye-protecting shields have included a face shield that a doctor or dentist would wear to prevent pathogens from getting into the user's mouth or eyes, or a transparent face shield portion of a motorcycle or snow-mobile helmet. Fogging that impairs vision is a common problem with such goggles, dive masks and eye-protecting shields. Examples of fog-prone displays have included hand-held GPS devices, hand-held radios, cellular phone devices, other portable electronic devices, wearable virtual reality headsets, wearable augmented reality headsets, and headsets comprising GPS devices, video cameras, and other instruments that may be used in cold-weather environments.

### SUMMARY OF THE INVENTION

In accordance with a first aspect of the invention, there is provided a portable, light attenuating electrochromic device, such as utilizes liquid crystal technology, adapted for heating to prevent fogging and for effectively attenuating impinging light despite colder weather operating conditions. The electrochromic device of this aspect of the invention comprises: first and second opposed substrates defining an enclosed space, each of the substrates having a conducting layer thereon and facing the other substrate. Further, the first substrate has a heating element bus bar system thereon for conducting current through the first conducting layer on the first substrate within a heating voltage range having an upper voltage limit and a lower voltage limit. Still further, the second substrate has a tint control bus bar system thereon for conducting current through the second conducting layer on the second substrate at first and second state tint voltages, each of the first and second state tint voltages being of a magnitude that is outside the heating voltage range upper and lower voltage limits. The device of this aspect of the invention further comprises: a liquid-crystal solution received within the enclosed space between the first and second opposed substrates, and first and second voltage supply power circuits. The first voltage supply power circuit is connected to the conducting layer of the first substrate via the heating element bus bar system, and the second voltage supply power circuit is connected to the conducting layer of the second substrate via the tint control bus bar system. Further, the device comprises means adapted for controlling battery power to the first and second voltage supply power circuits for heating the device during cold-weather operation to prevent fogging of the device and for attenuating light through the device to account for varying ambient lighting conditions despite colder weather operating conditions.

Preferably, the portable, light attenuating electrochromic device of this aspect of the invention is comprised, or operates, wherein the first state tint voltage of the second conducting layer of the second substrate is at a voltage above the upper heating voltage range, and further wherein the second state tint voltage of the second conducting layer of the second substrate is at a voltage below the lower heating voltage range.

This aspect of the invention provides a device which is capable of being heated to prevent fogging, and which is also capable of a change in tint to vary the amount of light transmittance to the device to accommodate varying ambient lighting conditions, all while providing an added feature and benefit of enabling preferably automated heating, as with a temperature sensing actuator, of the liquid-crystal material within the device to enable cold weather operability of the device beyond that otherwise possible without heating of the liquid-crystal material.

With this aspect, and other aspects of the invention, the heating of the lens for preventing fogging, for allowing cold-weather operability of the liquid crystal display technology, and for biasing the charge of the lens to enable tinting of the lens, may be accomplished by use of an indium-tin-oxide coating on the lens, as is known, or by use of carbon nano-tubes or other resistive heating technology.

It will be appreciated with the benefit of this disclosure by those of ordinary skill the art that there are various electronic means of delivering the two state voltages to the tint circuitry, apart from delivery of the different magnitude of heating voltage to the heating circuitry, such as by separate battery systems, or by deriving the differing voltages power from a single battery system, and further it will be appreciated with the benefit of this disclosure that the first state voltage for the tinting circuitry may be higher, or lower, than the heating voltage range experienced on the heating circuitry, whereas the opposing second state voltage for the tinting circuitry may also be higher, or lower, than the heating voltage range, as long as it is different than the first state tinting voltage, all without departing from the true spirit of the invention as claimed. Still further, it will be appreciated that, as long as there is a sufficient difference between the aforementioned two voltage states for the tint circuitry, both states may also be higher, or lower, than the highest, or lowest, heating circuit voltages, respectively, without departing from the scope and spirit of the invention claimed relating to the present invention.

In an aspect of the invention, the means adapted for controlling battery power to the first voltage supply power circuit for heating of the device to prevent fogging is continuously adjustable and preferably automated with a temperature sensing actuator which automatically heats the device as much as is needed to maintain the device in a sustainable temperature operating range depending on the temperature sensed. An added benefit of this feature to maintain cold weather operability would be that fogging of the device would also be automatically eliminated. Alternatively, the device could be configured to operate primarily in fog-prevention mode, where for example extreme cold-weather is not encountered but fogging is nevertheless a problem, and wherein operability of the device is dependent upon automated sensing and elimination of fogging by use of a dew-point detecting actuator. In such a situation, an override is provided for allowing continuous heating in the event of an extreme cold weather encounter.

In an alternative embodiment of the portable, light attenuating electrochromic device of this aspect of the invention, the means adapted for controlling battery power to the first voltage supply power circuit for heating the device to prevent fogging and enhance operability of the device despite colder weather operating conditions comprises a user-operable button operably connected to the device for tuning on and/or adjusting the amount of heat supplied to the device responsive to encountered fogging or unduly cold operating temperatures to allow continued effective operation of the light-attenuating features of the device. Another user-operable button could further be supplied which is operably connected to the device for biasing for allowing provision of, or elimination of, tint on demand by press of the button via connection through the means adapted for controlling battery power to the second voltage supply power circuit for attenuating light through the device to account for varying ambient lighting conditions.

Accordingly, still further, the portable, light attenuating electrochromic device of this aspect of the invention preferably further provides that the means adapted for controlling battery power to the first and second voltage supply power circuits is capable of varying the voltage applied to the respective circuits independently of each other in accordance with varying needs for heating and attenuation. This feature allows the device to function with respect to light attenuation even if heating is not required to prevent fogging or to continue effective operation because cold weather isn't encountered, but nevertheless such heating may be added independently if fogging and/or extreme cold weather is encountered.

In accordance with another aspect of the invention, a portable, light attenuating electrochromic device is provided wherein the liquid-crystal solution received within the enclosed space between the first and second opposed substrates further comprises a host solution having a guest dichroic dye dispersed therethrough to form a guest-host solution received between the substrates. Further, in accordance with this aspect of the invention, the means for controlling battery power to second voltage supply circuit for attenuating light through the device accounts for varying ambient lighting conditions by altering the polarization sensitivity and light transmission properties of the device by adjusting the orientation of the host solution and dichroic dye such that one polarization component of the impinging light can be variably absorbed at a different rate than another polarization component of the impinging light. As described in U.S. Pat. No. 6,239,778 to Palffy-Muhoray, this allows controllable sensitivity to polarized or non-polarized light which may or may not be automated through the use of a photocell actuator, allows controllable light transmittance and response time, allows a fail-safe device (that is, a device with high light transmittance when no electrical power is supplied), and allows a device which can accommodate varying colors and tints.

Thus, in accordance with this aspect of the invention, the portable, light-attenuating electrochromic device preferably provides light transmissivity that is relatively high when no electricity is produced by the second power circuit and that is relatively low when electricity is produced by the second power circuit. This feature allows the device to be used as a fail-safe high transmittance device in the event of a power failure, which is accomplished, as described in the '778 patent to Palffy-Muhoray, by having the director of the liquid crystal molecules align, through the use of alignment layers, in relatively parallel fashion to the majority of incoming light rays as depicted in FIG. 2A. Then, once an electric field is applied, the director changes from one that is relatively perpendicular to the substrate surfaces to one that is less perpendicular, or more parallel, as depicted in FIG. 2B, which causes the molecules of the dichroic dye to mimic the orientation of the liquid crystals and to absorb more light, resulting in decreased transmittance during an energized state.

The portable, light attenuating electrochromic device of either of these first two aspects of the invention may be used in either a goggle lens system wherein the device is held in a goggle frame which is adapted to engage a user's face and forms at least a partial enclosure around and in front of a user's eyes. Such a device may be used in a goggle frame that is fully enclosed with vents, or without vents, or alternatively such a device may be used in a partially enclosed vision screen, or other eyewear, more like a visor with contact of the users face across the eye-brow region of the user's face, or alternatively such a device may be used in some other portable vision screen lens system such as sunglasses, motor-cycle visors, medical visors, safety goggles, other eyewear and the like, any of which devices may be conducive to fogging to varying degrees, but which nevertheless are according to an aspect of the invention adapted for heating to prevent fogging impairment of vision of a user of the device.

Still further, this aspect of the portable, light attenuating electrochromic device of either of these aspects of the invention may be used in a visual display lens of a heads-up display in a goggle or vision screen, or in wearable virtual reality headset systems, or augmented reality headset systems, comprising an inner visual display lens. As is understood, the inner lens of such systems may comprise, together with a goggle or visor frame, at least a partial enclosure around the eyes and a part of the face of a user, such that these systems might likewise be conducive to fogging as a result of perspiration and condensation on an inner lens of the system, it being the case that such wearable systems may likewise be used in cold-weather operating environments, such as on a ski slope, during a military training exercise, or other gaming out of doors.

The subject matter of the present invention is particularly pointed out and distinctly claimed representing the scope of the invention in the concluding portion of this specification. However, both the organization and method of operation, together with further advantages and objects thereof, may best be understood by reference to the following descriptions taken in connection with accompanying drawings wherein like reference characters refer to like elements.

### BRIEF DESCRIPTION OF THE DRAWINGS

FIG. 1 is a schematic circuit diagram of a sample prior art circuit suitable for driving an electrochromic liquid-crystal cell;

FIG. 2a is a graphic side plan, or edge, view illustration of a prior art electrochromic liquid-crystal cell having a dichroic dye solution therein biased to a fail-safe, power-off, high transmittance state;

FIG. 2b is graphic side plan, or edge, view illustration of a prior art electrochromic liquid-crystal cell having a dichroic dye solution therein biased to a power-on, low transmittance, state;

FIG. 3 is a perspective graphic illustration of a prior art electrochromic liquid-crystal cell for illustrating the state operation of the cell and resulting transmissivity of light;

FIG. 4 is perspective graphic illustration of an electrochromic liquid-crystal cell adapted for heating to prevent fogging and to enhance cold-weather operability of the cell for illustrating the state operation of the cell in accordance with an aspect of the present invention;

FIG. 5 is a perspective graphic illustration and state diagram of an alternative embodiment electrochromic liquid-crystal cell having a dichroic dye solution therein and adapted for heating to prevent fogging and to enhance cold-weather operability of the cell in accordance with another aspect of the invention;

FIG. 6 is a graphic illustration of a circuit diagram and bus bar configuration graphic for controlling power to the cells of FIGS. 4 and 5 in accordance with an aspect of the invention;

FIG. 7 is graphic illustration of a goggle embodiment employing an electrochromic liquid-crystal-cell adapted for heating to prevent fogging and to enhance cold-weather operability of the cell in accordance with an aspect of the present invention;

FIG. 8 is graphic illustration of a vision screen embodiment employing an electrochromic liquid-crystal cell adapted for heating to prevent fogging and to enhance cold-weather operability of the vision screen in accordance with an aspect of the present invention; and

FIG. 9 is a graphic illustration of a virtual or augmented reality headset system employing an electrochromic liquid-crystal cell adapted for heating to prevent fogging and to enhance cold-weather operability of the device in accordance with an aspect of the present invention; and

FIG. 10 is a graphic illustration of a pair of eyewear, whether protective eyeglasses or prescription eyeglasses, employing an electrochromic liquid-crystal cell adapted for heating to prevent fogging and to enhance cold weather-operability of the device in accordance with an aspect of the present invention.

### DETAILED DESCRIPTION

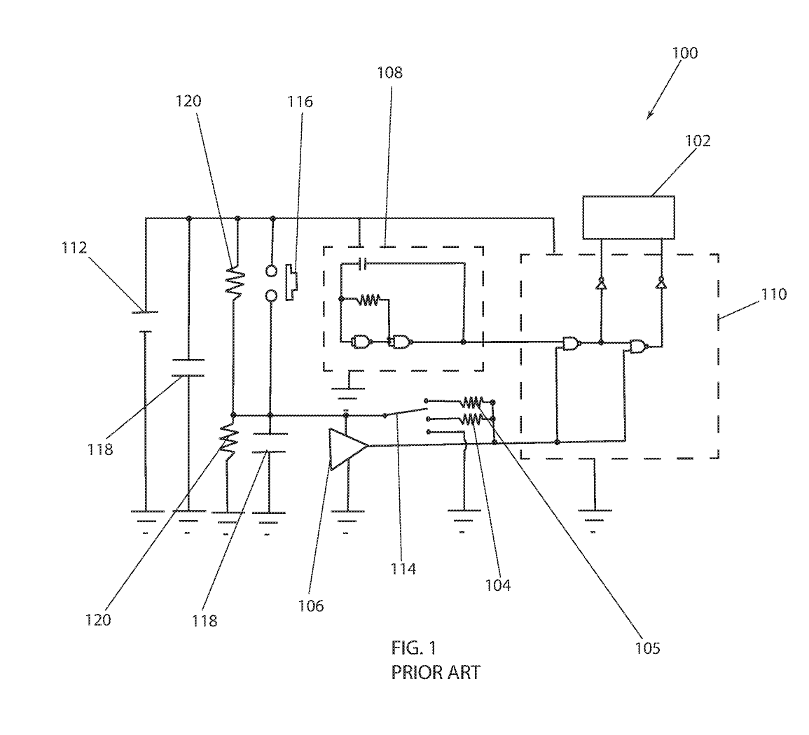

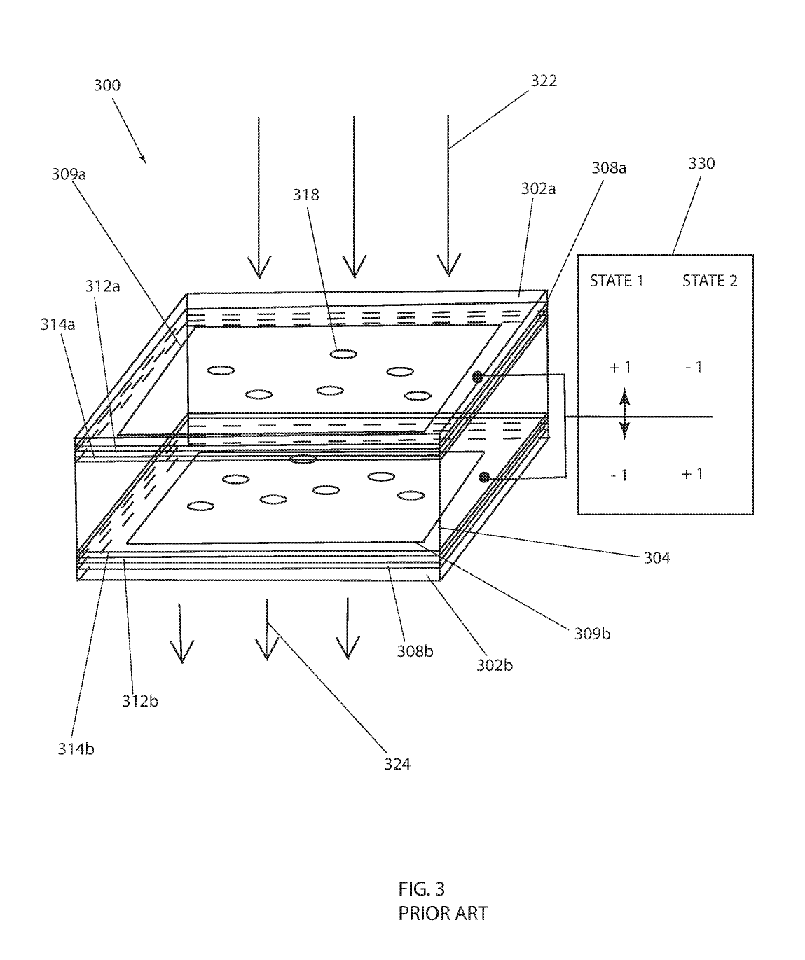

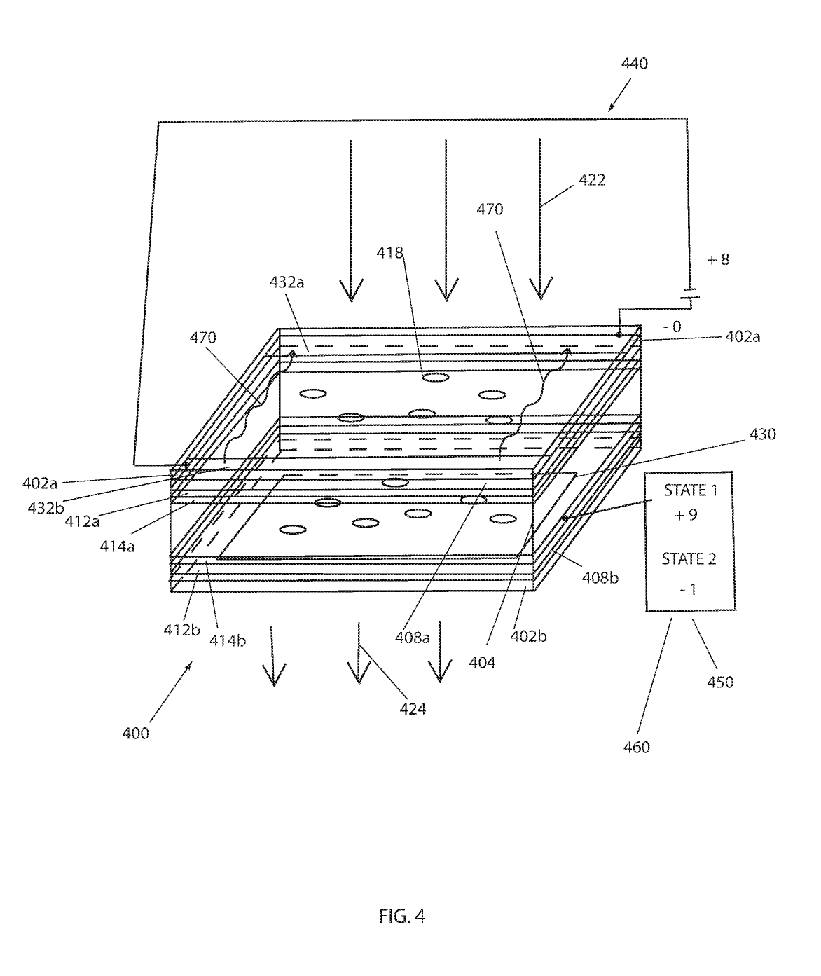

FIG. 1 shows a schematic diagram of a prior art circuit 100 for a standard electrochromic liquid-crystal cell 102 that may be used for part of the present invention for attenuating light transmission through the cell adapted for use in a pair of sunglasses, goggles, vision screen, virtual reality gaming or other portable VR system, augmented reality gaming or other portable AR system, or other portable electronic device. The circuit 100 is adapted from a circuit described in U.S. Pat. No. 5,015,086 to Okaue et al., which employs hysteresis (via resisters 104, 105) to aid in effective operation of the device during varying environmental lighting conditions as described in that patent. The circuit 100 is generally comprised of a voltage detecting circuit 106, an oscillating circuit 108, a liquid-crystal driving circuit 110, and other components (i.e., a switch 114 for set illumination, a touch switch 116 for powering the device to a forced illumination state, capacitors 118 for protecting the power source and delaying switching, and resistors 120 for voltage detection) as shown and described in connection with FIG. 3 of the OKaue et al. patent. The circuit 10 may be powered by a battery 12, or with solar power (not shown) as shown and described in the Okaue et al. patent. Further, an alternative embodiment of the circuit may likewise be employed as part of the present invention, as shown at FIG. 4 and described in the Okaue et al. patent, without departing from the true scope and spirit of the invention as claimed. Of course, it will be appreciated that other circuitry known in the art for driving an electrochromic cell for attenuating light, such as that shown and described in U.S. Pat. No. 6,239,778 to Palffy-Muhoray et al., may be employed as part of the present invention without departing from the true scope and spirt of the invention as claimed.

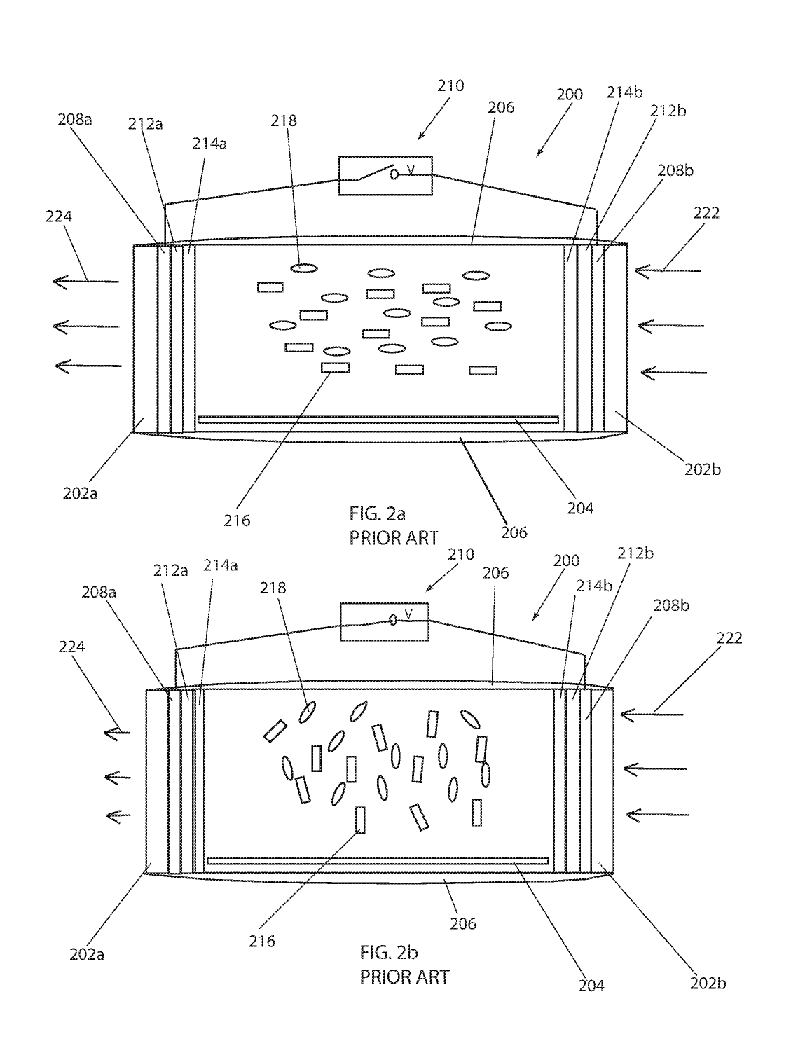

FIGS. 2a and 2b provide graphic illustrations of a prior art electrochromic liquid-crystal cell 200 having a dichroic dye solution therein as shown and described in the Palffy-Muhoray et al. patent, and which may be employed as part of the present invention. In each of the FIGS. 2a and 2b, corresponding to FIGS. 1a and 1b, respectively, of the Palffy-Muhoray et al. patent, there is provided a continuously electronically controllable light attenuating dichroic dye guest-host cell 200 comprising two substrates 202a, 202b having a separation 204 between them, allowing for a separation between the substrates of on the order of 5 to 20 μm's, and enclosed by a sealing material 206, such as epoxy. The substrates 202a, 202b are comprised of light-transmissive glass or plastic and are coated with resistive element conducting layers 208a, 208b. It will be appreciated that there are several different ways of applying heating material, such as Indium Tin Oxide (ITO), carbon nano-wires, or other resistive heating material, to the substrates 202a, 202b, including commonly known methods of ion sputtering, coating, vacuum deposited coating, spraying, adhesive, adhesive backed and other methods. The resistive element conducting layers 208a, 208b are connected to a power circuit 210 having a variable voltage supply. An optional passivation layer 212a, 212b may also be employed to minimize the possibility of short circuiting, and there is also provided an alignment layer 214a, 214b to serve further as a passivation layer.

The device 200 of FIGS. 2a and 2b is shown having enclosed therein a guest-host solution as shown and described in the Palffy-Muhoray et al. patent comprised of a dichroic dye 216 in a liquid-crystal host material 218. Dichroic dye 216 may employ either positive or negative dichroism and may be comprised preferably of any chemical-, temperature-, and UV-stable organic molecule or mixture whose absorption of polarized light strongly depends on the direction of polarization relative to the absorption dipole in the molecule, all as described in the Palffy-Muhoray et al. patent.

In a resting state, preferably, and as shown in FIG. 2a, the cell 200 is preferably biased by its alignment layers 214a, 214b such that peak light transmission is achieved as shown, wherein the dichroic dye 216 and liquid-crystal host material 218 are shown aligned perpendicular to the substrates 202a, 202b, thus allowing a maximum amount of light to pass through the cell as represented by arrows 222, 224. As shown in FIG. 2b, in an active state, wherein the dichroic dye 216 and liquid-crystal host material 218 are shown to be aligned more parallel to the substrates 202a, 202b, responsive to the charge in the conductive elements 208a, 208b, less light is allowed to pass through the cell 200 as represented by arrows 222, 224. It will be appreciated by those of ordinary skill in the art that the aforementioned states produce the light attenuation characteristics described depending upon a negative or positive dichroism of the dye 216, the alignment layers' 214a, 214b characteristics, and any charge applied through the conductive elements 208a, 208b, all as described in the Palffy-Muhoray et al. patent and known in the art, and it will be further appreciated that these factors and elements may be alternatively employed in such a way as to produce minimal light transmissivity through the cell 200 in a resting state, as may be for example beneficial for use in a welding helmet, without departing from the true scope and spirit of the invention as claimed. Thus, in an eyeglasses, goggles or other lens application, a “fail safe” system is comprised of maximum light transmissivity during a resting, or off, state of the device, whereas in a device where the cell's 200 going dark is not problematic, or even beneficial, in an off state, a “fail safe” for such a device would comprise minimal light transmissivity during resting, or off, state of the device.

The eye shield substrates 202a, 202b may be selected from any of a number of materials, such as optically-transparent polycarbonate, other plastic, tempered glass, and the like, that are rigid and durable enough to screen a user's eyes from such things as snowfall, rain, wind, or even shrapnel for a ballistics-rated system, or other relatively small airborne particles in the user's environment. Further, to function properly as liquid-crystal cells 200 per the present invention, the materials selected must be sufficiently rigid to retain a consistent distance between the anterior and posterior substrate members comprising the cell.

Referring now to FIG. 3, another prior art electrochromic liquid-crystal cell 300 is shown, which is substantially identical to the cell 200 of FIGS. 2a and 2b, except that the cell 300 does not include the dichroic dye like the cell 200. Thus, cell 300 comprises substrates 302a, 302b, resistive conductive elements 308a, 308b (including a continuous rectangular bus bars 309a, 309b), passivation layers 312a, 312b and alignment layers 314a, 314b. A separation/separator is illustrated at 304. The cell 300 is shown in State 1, in this case with minimal transmissivity of light being illustrated since liquid crystals 318 are shown oriented so as to block light transmission, and with the polarity of the device being indicated in the table to the right. Thus, the arrows 322 illustrate light entering into the cell 300, and arrows 324 illustrate significantly less light leaving the other side of the cell. Of course, switching the state of the device to State 2, would alter the directional orientation of the liquid crystals 318 and allow more light to transfer through the cell 300, and this state change is effected by altering the relative polarity of the cell as indicated in the State table 330.

Referring now to FIG. 4, a perspective graphic illustration of an electrochromic liquid-crystal cell 400 adapted for heating to prevent fogging and to enhance cold-weather operability is shown in accordance with an aspect of the invention. Similar to the construction of cells 200a, 200b, and 300, cell 400 comprises substrates 402a, 402b, resistive conductive layers 408a, 408b, optional passivation layers 412a, 412b, alignment layers 414a, 414b, and spacer represented by 404. However, in accordance with the present invention, bus bars 430, 432a and 432b, including a rectangular continuous tint bus bar 430 and opposing non-continuous heat bus bars (i.e., upper and lower, or left and right, bus bar strips) 432a, 432b, are provided. Further, heating power circuitry 440 is provided with a power application regimen resulting in state configuration 450 for the cell 400 as illustrated in the State table 450 of FIG. 4.

Thus, as shown in FIG. 4, in State 1, 9 volts of electricity are passed from a tint power system 460 through the continuous tint bus bar 430 to create a polarity differential between the high, +8, voltage heating element portion of the cell 400, in order to bias the liquid crystals of the cell to a horizontal state, which blocks more light from passing through the cell as indicated by arrows 422, 424 (arrows 424 are smaller than arrows 422, indicating less light is passing through the cell 400). As power transmits through the conductive layer 408a, it encounters the resistance of the conductive material, and this results in a voltage drop across the conductive layer as illustrated by wavy arrows 470. The voltage drop is shown as 8 volts (from +8 to −0 volts). Thus, to change state of the tint bus bar and corresponding conductive material 408b in order to alter the orientation of liquid crystals 418 to allow greater light transmissivity, the tint power 460 system generates a −1 voltage, which is a sufficiently differential voltage relative to the low power state of the heating circuit 440 and bus bars 432a, 432b. Thus, in this manner, not only does the system 400 provide for the required state change to allow varying the transmissivity of light through the cell 400, but also the liquid crystal solution 418 is warmed sufficiently to provide enhanced operability of the cell despite colder-weather operating temperatures.

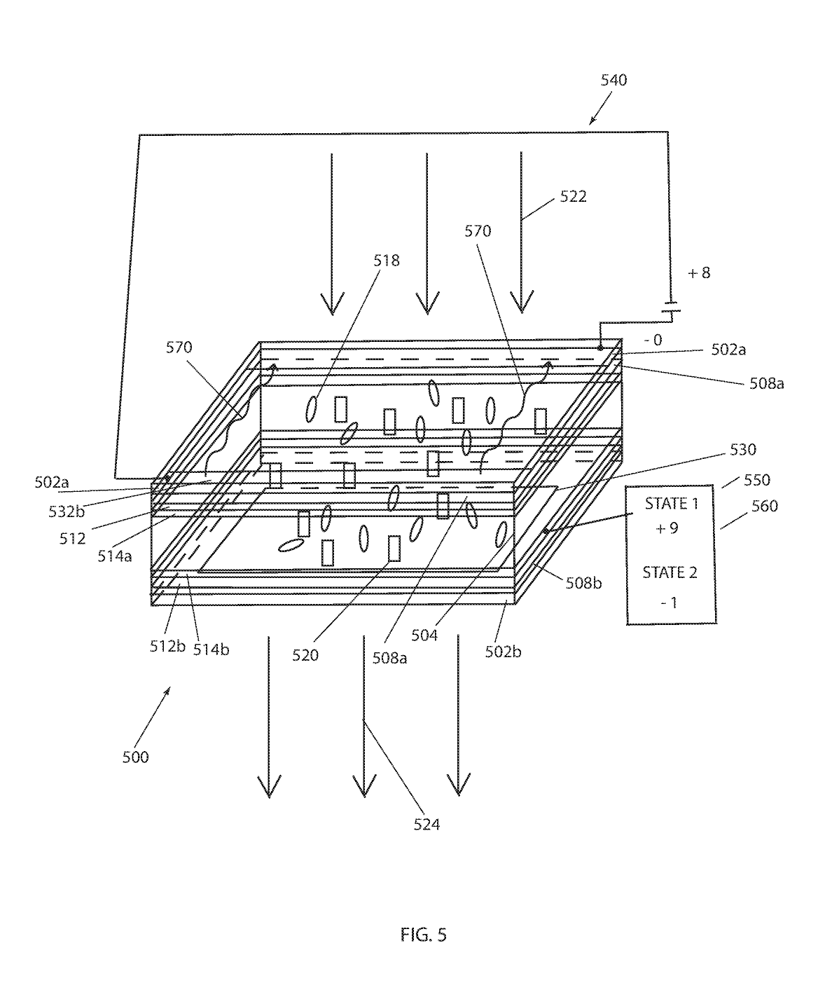

Referring now to FIG. 5, a perspective graphic illustration of an alternate electrochromic liquid-crystal cell 500 adapted for heating to prevent fogging and to enhance cold-weather operability is shown. Similar to the construction of cells 200a, 200b, 300 and 400, cell 500 comprises substrates 502a, 502b, resistive conductive layers 508a, 508b, optional passivation layers 512a, 512b, alignment layers 514a, 514b, and spacer 504. However, in accordance with the present invention, bus bars 530, 532, including a rectangular continuous (rectangular picture-frame shaped, or alternatively annular or other continuous shape) tint bus bar 530 and opposing non-continuous heat bus bars (i.e., upper and lower, or left and right, bus bar rectangular strips) 532a, 532b, are provided. Of course, the bus bars may take the shape necessary to conform to the contours of the edges of the cell, whether it be rectangular, circular, oval, oblong or otherwise as shown in other Figures hereof without departing from the true scope and spirit of the invention. Further, heating power circuitry 540 is provided with a power application regimen resulting in state configuration 550 for the cell 500 as illustrated in the State table 550 of FIG. 5. Unlike cells 300 and 400, cell 500 includes both liquid crystals 518 and dichroic dye 520 to enable to tune sensitivity of the device to light polarization.

Thus, as shown in FIG. 5, in State 2, −1 volts of electricity are passed from a tint power system 560 through the continuous tint bus bar 530 to create a polarity differential between the low, −0, voltage heating element portion of the cell 500, in order to bias the liquid crystals 518 and associated dichroic dye 520, of the cell to a perpendicular state (relative to the substrates 502a, 502b, which allows more light to pass through the cell as indicated by arrows 522, 524 (arrows 524 are about the same size as arrows 522, indicating more light is passing through the cell 500 than in the case of cell 400 shown in FIG. 4). As electric power transmits through the conductive layer 508a, it encounters the resistance of the conductive material, and this results in a voltage drop and generation of heat across the conductive layer as illustrated by wavy arrows 570. The voltage drop is shown as 8 volts (from +8 to −0 volts). Thus, to change state of the tint bus bar and corresponding conductive material 508b in order to alter the orientation of liquid crystals 518 to allow lesser light transmissivity, the tint power 560 system generates a +9 voltage, which is sufficient differential voltage relative to the high power state of the heating circuit 540 and bus bars 532a, 532b. Thus, in this manner, not only does the system 500 provide for the required state change to allow varying the transmissivity of light through the cell 500, but also the liquid crystal 518 and dichroic dye 520 solution is warmed sufficiently to provide enhanced operability of the cell despite colder-weather operating temperatures.

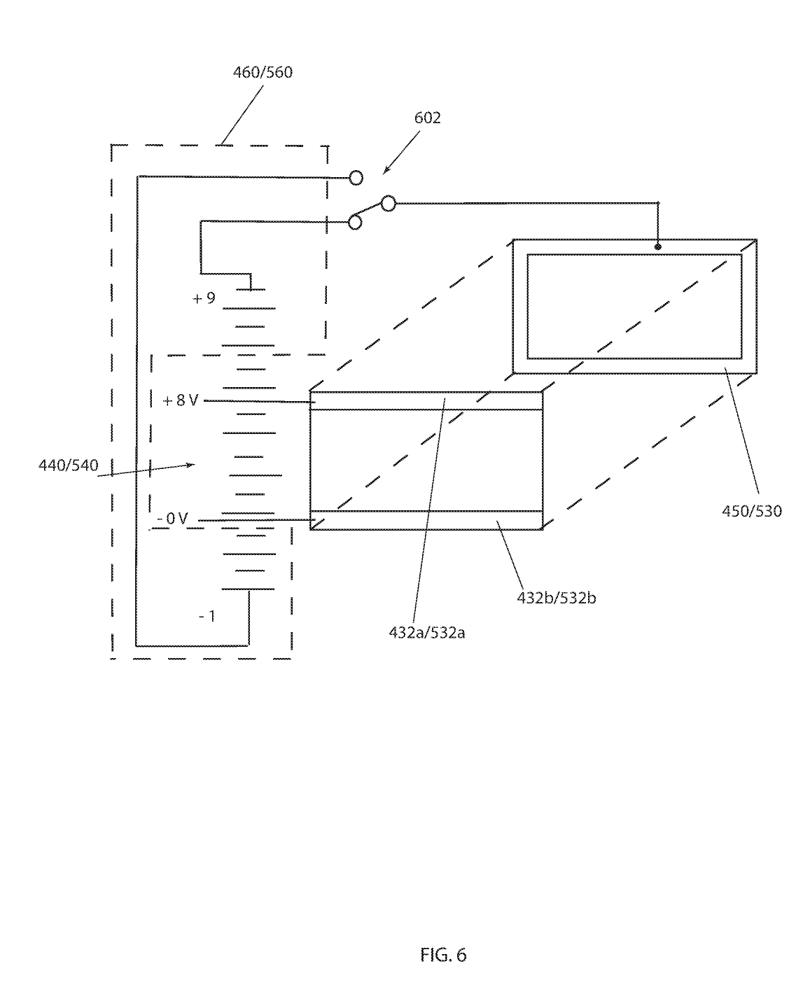

Referring to FIG. 6, a graphic illustration of a circuit diagram and bus bar configuration graphic for controlling power to the cells of FIGS. 4 and 5 in accordance with an aspect of the invention is provided. As described previously in connection with cells 400 and 500, bus bars 432a/532a, 432b/532b, and 430/530 are shown together with a simple circuit, comprising both a tint power circuit 460/560 and a heating power circuit 440/540, for powering both the tint control features of the invention and the heating features of the invention. A switch 602 is used to change state for the tint control power circuit 460/560, and each of the systems may be controlled with an on/off button, as is known in the art, or other automated dew point calculating and/or light sensing means known in the art. As appreciated by those skilled in the art given the teachings herein, the tint power control circuit 460/560 may comprise hysteresis and/or protection circuitry as taught in the Okaue et al. patent, and the heating control circuit 440/540 may comprise power control similar to that shown and described in U.S. Pat. No. 8,566,962 for PWM Heating System for Eye Shield by Cornelius. In the Cornelius patent, a system of multiple channels is disclosed for controlling power to each of the channels of an eye shield using PWM, and such a control system may be advantageously used to create the bifurcated tint power control circuit 460/560 and heating power control circuit 440/540. Or alternatively, the power to the two control systems, circuit 460/560 and circuit 440/540, may be accomplished by other means of directing differential power to different loads as known in the art of electronics without departing from the true scope and spirit of the invention as claimed.

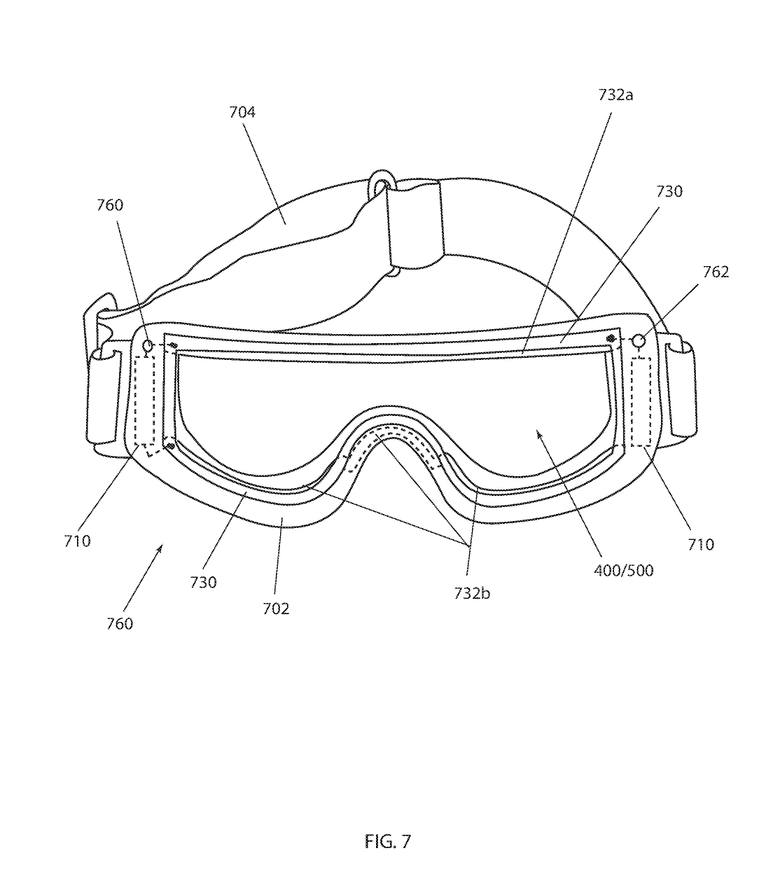

Referring to FIG. 7, in a goggle eye shield 700, a goggle frame 702 holds the cells 400, 500, batteries 710, tint control button 762 and heating power control button 760. It will be appreciated that other methods of starting the systems may be implemented in accordance with that understood in the art, such as automated methods using light and/or humidity sensors. Note from FIG. 7 that the cells 400, 500 comprise a tint control bus bar 730 and related power system (the same as that described relative to either cell 400 or cell 500), as well as a heater control bus bar 732a (upper bus bar), 732b (lower bus bar). Thus, when a user of the goggle eye shield 700 encounters fogging, he or she is enabled in de-fogging the eye shield by pressing heating power button 760 (or otherwise the heating power system is activated as with an automated program based on a temperature/humidity sensor or otherwise), and he or she is also enabled in adjusting the tint by pressing tint control button 762 (or otherwise the tint is able to be automatically changed relative to ambient lighting conditions). Further, the user is enabled in using such a device in very cold weather, since the heater control power system keeps the liquid-crystal material warmed to be able to flow more freely and thus achieve state changes to adapt to changing ambient lighting conditions.

While cells 400, 500 of goggle eye shield 700 are rigid, the frame 702 must also be able generally to conform to the user's head and face with the eye shield 700 preferably being retained in a frame that holds the eye shield around its periphery. Also, the eye shield 700 is held an appropriate distance from the user's face, so as to form an enclosed space around and in front of the user's eyes, with the use of a conventional goggle strap 704. Thus, the goggle frame 702 typically provides a semi-permeable seal between the user's face and the rest of the goggle. Materials used for the various eye shields 700 employed with the present invention should also be resistant to shattering, cracking or otherwise breaking as necessary for the particular purpose for which they are chosen and as is known to those of ordinary skill in the art.

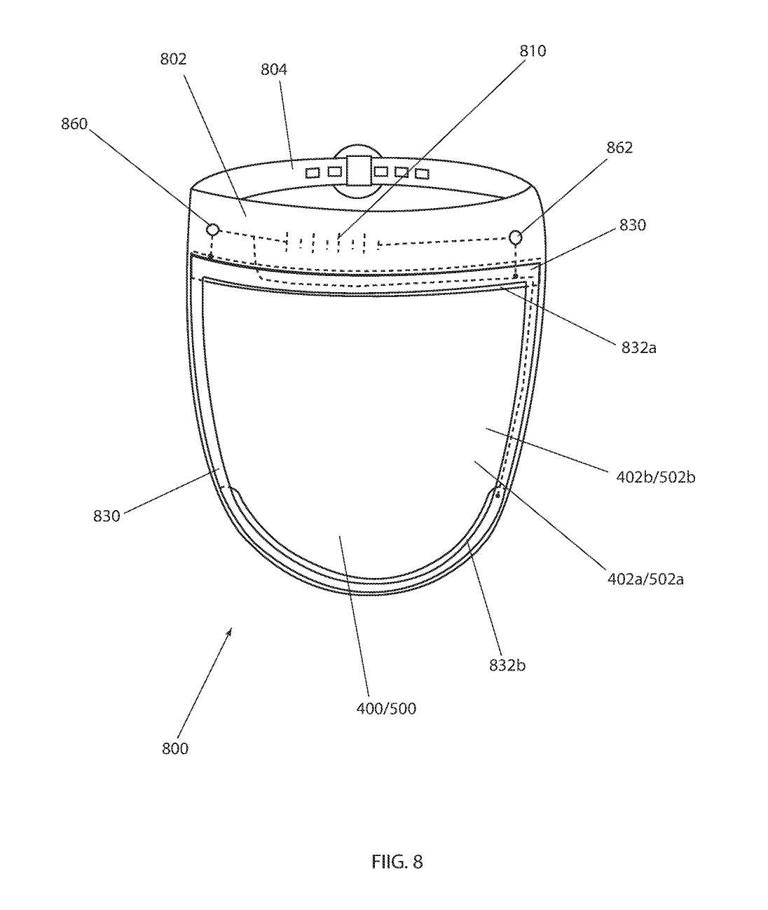

Referring to FIG. 8, in an eye shield visor 800, such as a medical visor (or similar to that adapted for a motorcycle helmet), a frame 802 holds the cells 400, 500, battery 810, tint control button 862 and heating power control button 860. It will be appreciated that other methods of starting the systems may be implemented in accordance with that understood in the art, such as automated methods using light and/or humidity sensors. Note from FIG. 8 that the cells 400, 500 comprise a tint control bus bar 830 and related power system (the same as that described relative to either cell 400 or cell 500), as well as a heater control bus bar 832a (upper bus bar) and 832b (lower bus bar). Thus, when a user of the eye shield 800 encounters fogging, he or she is enabled in de-fogging the eye shield by pressing heating power button 860 (or otherwise the heating power system is activated as with an automated program based on a temperature/humidity sensor or otherwise), and he or she is also enabled in adjusting the tint by pressing tint control button 862 (or otherwise the tint is able to be changed, for example automatically, relative to ambient lighting conditions). Further, the user is enabled in using such a device 800 in very cold weather, since the heater control power system keeps the liquid-crystal material warmed to be able to flow more freely and thus achieve state changes to adapt to changing ambient lighting conditions.

While cells 400, 500 of goggle eye shield 800 are rigid, the frame 802 must also be able generally to conform to the user's head and face with the eye shield 800 preferably being retained in a frame that holds the eye shield around its periphery, or at least along the top of the eye shield as shown. Also, the eye shield 800 is held an appropriate distance from the user's face, so as to form at least a partially enclosed space around and in front of the user's eyes, with the use of a conventional adjustable band 804. Materials used for the various eye shields 800 employed with the present invention should also be resistant to shattering, cracking or otherwise breaking as necessary for the particular purpose for which they are chosen and as is known to those of ordinary skill in the art.

The eye shield 800 substrates 402a, 402b (502a, 502b) are preferably made from a rigid plastic, or glass, material, and in the case of a visor or medical full face eye shield 800, the substrate 402a/502a, 402b/502b would likewise be selected of a somewhat more rigid plastic, or glass, material that is sufficiently light weight, but also sufficiently rigid to allow durable and repeated positioning of the eye shield in place to protect the user's eyes. Selection of the eye shield substrates will preferably be of a material that is smooth to the touch, both on its inner (posterior) surface and its outer (anterior) surfaces and which is adapted to form a bond with the selected heating material, bus bars and sealing material for forming the enclosure for the liquid-crystal host material and any dye material in accordance with aspects of the invention. Eye shield substrate materials are well known to those of ordinary skill in the art, and the selection of any type of optically-transparent eye shield substrate shall fall within the scope of the claims appended hereto.

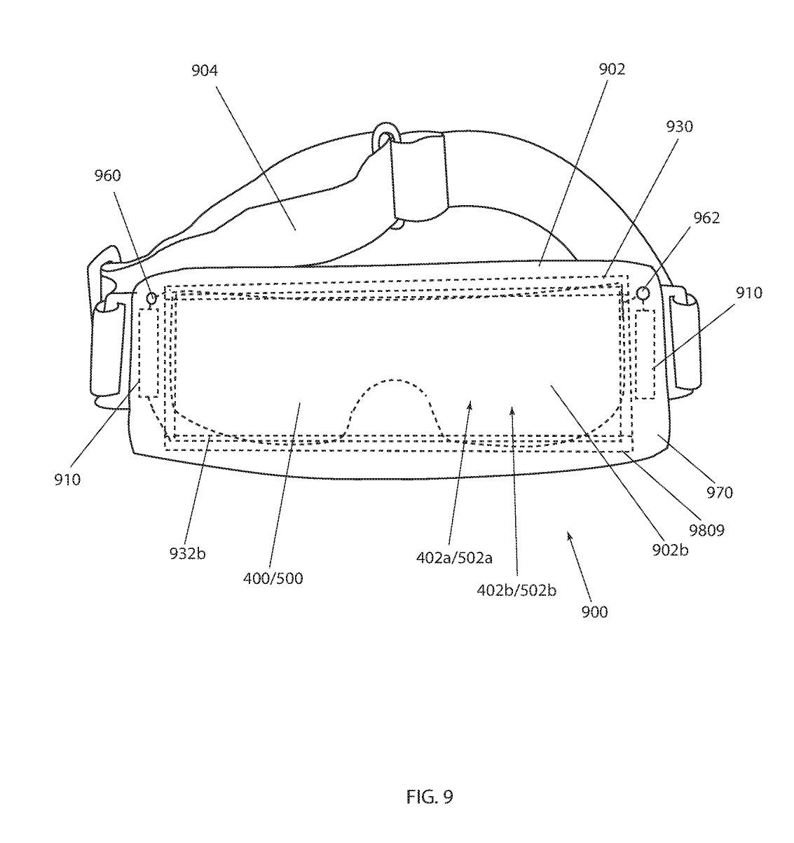

Referring to FIG. 9, in a virtual reality (VR), or augmented reality (AR), system 900, such as an available device for holding a person's cellular phone, or other video playing device, up to a user's eyes to create the appearance of a dynamic, virtual, 3d, real-time virtual, or augmented, reality view, a frame 902 holds the cells 400, 500, batteries 910, tint control button 962 and heating power control button 960. It will be appreciated that other methods of starting the systems may be implemented in accordance with that understood in the art, such as automated methods using light, temperature and/or humidity sensors. Note from FIG. 9 that the cells 400, 500 comprise a tint control bus bar 930 and related power system (the same as that described relative to either cell 400 or cell 500), as well as a heater control bus bar 932a (upper bus bar) and 932b (lower bus bar). Thus, when a user of the VR/AR system 900 encounters fogging, he or she is enabled in de-fogging the eye shield by pressing heating power button 960 (or otherwise the heating power system is activated as with an automated program based on a temperature/humidity sensor or otherwise), and he or she is also enabled in adjusting the tint by pressing tint control button 962 (or otherwise the tint is able to be changed, for example automatically, relative to ambient or programmed lighting conditions). Further, the user is enabled in using such a device 900 in very cold weather, since the heater control power system keeps the liquid-crystal material warmed to be able to flow more freely and thus achieve state changes to adapt to changing ambient lighting conditions. A front cover 970 may be implemented either as part of an integrated AR/VR system 900, or alternatively, the cover 970 may be removable to allow insertion of a smart phone or other video gaming device (not shown) into a receptacle 980 defined around and anteriorly of the cell 400/500. After insertion of the removable smart phone, etc., the cover 970 may be snapped back into place to cover and protect the smart phone.

While cells 400, 500 of VR/AR system 900 are rigid, the frame 902 must also be able generally to conform to the user's head and face with the VR/AR system 900 preferably being retained in a frame that holds the eye shield around its periphery, or at least along the top of the eye shield as shown. Also, the system 900 is held an appropriate distance from the user's face, so as to form at least a partially enclosed space around and in front of the user's eyes, with the use of a conventional adjustable strap 904. Materials used for the VR/AR system 900 frame and cells 400/500 employed with the present invention should be resistant to shattering, cracking or otherwise breaking as necessary for the particular purpose for which they are chosen and as is known to those of ordinary skill in the art. Frame 902 also holds batteries 910 to provide power to the system's needs.

The system 900 substrates 402a, 402b (502a, 502b) are preferably made from a rigid plastic, or glass, material, and in the case of a VR/AR system 900, the substrate 902a, 902b would likewise be selected of a somewhat more rigid plastic, or glass, material that is sufficiently light weight, but also sufficiently rigid to allow durable and repeated positioning of the eye shield in place to use the VR/AR system. Selection of the eye shield substrates 402a, 402b (502a, 502b) will preferably be of a material that is smooth to the touch, both on its inner (posterior) surface and its outer (anterior) surfaces and which is adapted to form a bond with the selected heating material, bus bars and sealing material for forming the enclosure for the liquid-crystal host material and any dye material in accordance with aspects of the invention. Eye shield substrate materials are well known to those of ordinary skill in the art, and the selection of any type of optically-transparent eye shield substrate shall fall within the scope of the claims appended hereto.

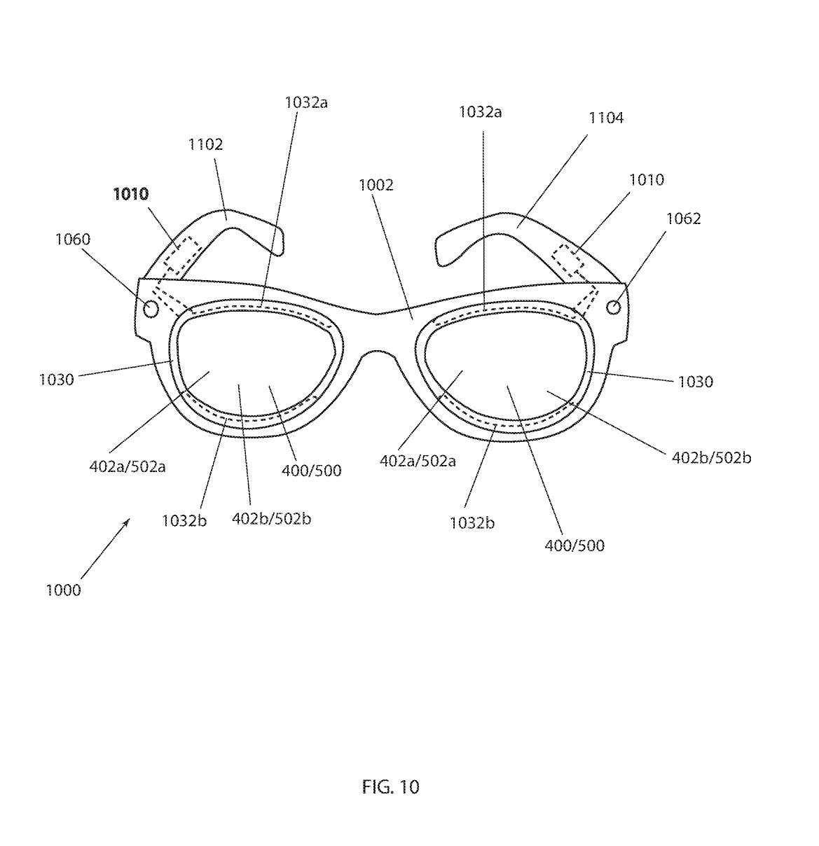

Referring now to FIG. 10, there is shown a graphic illustration of a pair of eyewear 1000, whether protective eyeglasses or prescription eyeglasses, employing electrochromic liquid-crystal cells 400/500 adapted for heating to prevent fogging and to enhance cold weather-operability of the device in accordance with an aspect of the present invention. In such an eyewear 1000, a frame 1002 holds the cells 400, 500, batteries 1010, tint control button 1062 and heating power control button 1060. It will be appreciated that other methods of starting the systems may be implemented in accordance with that understood in the art, such as automated methods using light and/or humidity sensors. Note from FIG. 10 that the cells 400, 500 comprise a tint control bus bar 1030 and related power system (the same as that described relative to either cell 400 or cell 500), as well as a heater control bus bar 1032a (upper bus bar) and 1032b (lower bus bar). Thus, when a user of the eyewear 1000 encounters fogging, he or she is enabled in de-fogging the eyewear by pressing heating power button 1060 (or otherwise the heating power system is activated as with an automated program based on a temperature/humidity sensor or otherwise), and he or she is also enabled in adjusting the tint by pressing tint control button 1062 (or otherwise the tint is able to be changed, for example automatically, relative to ambient lighting conditions). Further, the user is enabled in using such a device 1000 in very cold weather, since the heater control power system keeps the liquid-crystal material warmed to be able to flow more freely and thus achieve state changes to adapt to changing ambient lighting conditions.

While cells 400, 500 of the eyewear 1000 are rigid, the frame 1002 must also be able generally to conform to the user's head and face, using standard eyeglasses temples 1102, 1104 with the cells 400/500 preferably being retained in the 1002 frame that holds the cells around their periphery. Also, the eyewear 1000 is held an appropriate distance from the user's face and eyes. A leash, strap, or band (not shown) may also be used to help retain the eyewear 1000 on the user's face during strenuous activity. Materials used for the various eye shields employed with the present invention should also be resistant to shattering, cracking or otherwise breaking as necessary for the particular purpose for which they are chosen and as is known to those of ordinary skill in the art.

The substrates 402a, 402b (502a, 502b) of the present invention are preferably made from a rigid plastic, or glass, material, however a material and thickness must be selected that is sufficiently light weight, but also sufficiently rigid to allow durable and repeated positioning of the eye shield in place to protect the user's eyes. Selection of the eye shield substrates will preferably be of a material that is smooth to the touch, both on its inner (posterior) surface and its outer (anterior) surfaces and which is adapted to form a bond with the selected heating material, bus bars and sealing material for forming the enclosure for the liquid-crystal host material and any dye material in accordance with aspects of the invention. Eye shield substrate materials are well known to those of ordinary skill in the art, and the selection of any type of optically-transparent eye shield substrate shall fall within the scope of the claims appended hereto.

The bus bars of any of the system of the present invention may be applied using known methods of silver ink, metal foil in contact with the conductive resistive elements of the various systems described, or other known method of creating a suitable bus bar.

While preferred embodiments of the present invention have been shown and described, it will be apparent to those skilled in the art that many changes and modifications may be made without departing from the invention in its broader aspects. For example, it will be appreciated that one of ordinary skill in the art may mix and match the various components of the various embodiments of the invention without departing from the true spirit of the invention as claimed. The appended claims are therefore intended to cover all such changes and modifications as fall within the true spirit and scope of the invention.

1. A portable, light attenuating electrochromic device adapted for heating to prevent fogging and for effectively attenuating impinging light despite colder weather operating conditions comprising:
a. first and second opposed substrates defining an enclosed space, each said substrate having a conducting layer thereon and facing the other substrate, wherein said first substrate has a heating element bus bar system thereon for conducting current through the first conducting layer on said first substrate within a heating voltage range having an upper voltage limit and a lower voltage limit, and wherein said second substrate has a tint control bus bar system thereon for conducting current through the second conducting layer on said second substrate at first and second state tint voltages, each of the first and second state tint voltages being of a magnitude that is outside the heating voltage range upper and lower voltage limits;
b. a liquid-crystal solution received within the enclosed space between said first and second opposed substrates;
c. first and second voltage supply power circuits, said first voltage supply power circuit connected to the conducting layer of said first substrate via the heating element bus bar system, said second voltage supply power circuit connected to the conducting layer of said second substrate via the tint control bus bar system; and
d. means adapted for controlling battery power to said first and second voltage supply power circuits for heating the device during cold-weather operation to prevent fogging of the device and for attenuating light through the device to account for varying ambient lighting conditions despite colder weather operating conditions.

2. The portable, light attenuating electrochromic device of claim 1, wherein the first state tint voltage of the second conducting layer of said second substrate is at a voltage above the upper heating voltage range, and wherein the second state tint voltage of the second conducting layer of said second substrate is at a voltage below the lower heating voltage range.

3. The portable, light attenuating electrochromic device of claim 1, wherein said means adapted for controlling battery power to said first and second voltage supply power circuits for heating the device to prevent fogging and for attenuating light through the device to account for varying ambient lighting conditions despite colder weather operating conditions comprises a plurality of user-operable buttons operably connected to the device.

4. The portable, light attenuating electrochromic device of claim 2, wherein said means adapted for controlling battery power to said first and second voltage supply power circuits is capable of varying the voltage applied to the respective circuits independently in accordance with varying needs for heating and attenuation.

5. The portable, light attenuating electrochromic device of claim 4, wherein said liquid-crystal solution received within the enclosed space between said first and second opposed substrates further comprises a host solution having a guest dichroic dye dispersed therethrough to form a guest-host solution received between said substrates, and wherein said means for controlling battery power to said second voltage supply circuit for attenuating light through the device accounts for varying ambient lighting conditions by altering the polarization sensitivity and light transmission properties of the device by adjusting the orientation of said host solution and dichroic dye such that one polarization component of the impinging light can be variably absorbed at a different rate than another polarization component of the impinging light.

6. The portable, light attenuating electrochromic device of claim 5, wherein light transmissivity is relatively high when no electricity is produced by said second power circuit and relatively low when electricity is produced by said second power circuit.

7. The portable, light attenuating electrochromic device of claim 1, used in one of a goggle lens, a portable vision screen lens, and an eyeglasses lens adapted for heating to prevent fogging impairment of vision of a wearer of the lens.

8. The portable, light attenuating electrochromic device of claim 1, used in a visual display of a wearable headset display device adapted for one of a virtual reality display and an augmented reality display and adapted for heating of the visual display to prevent fogging impairment of visibility of the display by a user of the electronic device.

9. An electronically-operable, portable, light attenuating liquid-crystal device adapted for variable heating to prevent fogging and for effectively attenuating impinging light despite cold weather conditions comprising:
a. first and second opposed substrates defining an enclosed space, each said substrate having a conducting layer thereon and facing the other substrate, wherein said first substrate has a heating element bus bar system thereon for conducting current through the first conducting layer on said first substrate within a heating voltage range having an upper voltage limit and a lower voltage limit, and wherein said second substrate has a tint control bus bar system thereon for conducting current through the second conducting layer on said second substrate at at least a first state tint voltage above the upper voltage limit of the heating voltage range and a second state tint voltage below the lower voltage limit of the heating voltage range;
b. a liquid-crystal solution received within the enclosed space between said first and second opposed substrates;
c. first and second voltage supply power circuits, said first voltage supply power circuit being continuously variable and connected to the conducting layer of said first substrate via its corresponding bus bar system to variably alter the heating of said first substrate according to cold-temperature needs, said second voltage supply power circuit connected to the conducting layer of said second substrate via its corresponding bus bar system to allow change of voltage supplied to the conducting layer of said second substrate to alter the light attenuation of the device; and
d. means adapted for controlling battery power to said first and second voltage supply power circuits for heating said device to prevent fogging and for attenuating light through the device to account for varying ambient lighting conditions despite colder weather operating conditions.

10. The electronically-operable, portable, light attenuating liquid-crystal device of claim 9, wherein said means adapted for controlling battery power to said first and second voltage supply power circuits for heating said device to prevent fogging and for attenuating light through the device to account for varying ambient lighting conditions despite colder weather operating conditions comprises a plurality of user-operable buttons operably connected to said device.

11. The electronically-operable, portable, light attenuating liquid-crystal device of claim 10, wherein said means adapted for controlling battery power to said first and second voltage supply power circuits is capable of varying the voltage applied to the respective circuits independently in accordance with varying needs for heating and attenuation.

12. The electronically-operable, portable, light attenuating liquid-crystal device of claim 11, wherein said liquid-crystal solution received within the enclosed space between said first and second opposed substrates further comprises a host solution having a guest dichroic dye dispersed therethrough to form a guest-host solution received between said substrates, and wherein said means for controlling battery power to said second voltage supply circuit for attenuating light through the device accounts for varying ambient lighting conditions by altering the polarization sensitivity and light transmission properties of the device by adjusting the orientation of said host solution and dichroic dye such that one polarization component of the impinging light can be variably absorbed at a different rate than another polarization component of the impinging light.

13. The electronically-operable, portable, light attenuating liquid-crystal device of claim 12, wherein light transmissivity is relatively high when no electricity is produced by said second power circuit and relatively low when electricity is produced by said second power circuit.

14. The electronically-operable, portable, light attenuating liquid-crystal device of claim 9, used in one of a goggle lens and a vision-screen lens adapted for heating to prevent fogging impairment of vision of a wearer of the lens.

15. The electronically-operable, portable, light attenuating liquid-crystal device of claim 9, used in a visual display of a wearable headset display device adapted for one of a virtual reality display and an augmented reality display and adapted for heating of the visual display to prevent fogging impairment of visibility of the display by a user of the electronic device.

16. An electronically-operable, portable, variable, light attenuating liquid-crystal device adapted for heating to prevent fogging and for effectively attenuating impinging light despite colder weather conditions comprising:
a. first and second opposed substrates defining an enclosed space, each said substrate having a conducting layer thereon and facing the other substrate, wherein said first substrate has a heating element bus bar system thereon for conducting current through the first conducting layer on said first substrate within a heating voltage range having an upper voltage limit and a lower voltage limit, and wherein said second substrate has a tint control bus bar system thereon for conducting current through the second conducting layer on said second substrate at a first state tint voltage above the upper voltage limit of the heating voltage range and a second state tint voltage below the lower voltage limit of the heating voltage range;
b. a liquid-crystal solution received within the enclosed space between said first and second opposed substrates comprising a host solution having a guest dichroic dye dispersed therethrough to form a guest-host solution received between said substrates;
c. first and second variable voltage supply power circuits, said first voltage supply power circuit connected to the conducting layer of said first substrate via its corresponding bus bar system to alter the heating of said first substrate according to cold-temperature needs, said second voltage supply power circuit connected to the conducting layer of said second substrate via its corresponding bus bar system to alter the polarization sensitivity and light transmission properties of the cell by adjusting the orientation of the liquid-crystal solution and dichroic dye such that one polarization component of the impinging light can be variably absorbed at a different rate than another polarization component of the impinging light; and
d. means adapted for controlling battery power to said first and second voltage supply power circuits for heating said device to prevent fogging and for attenuating light through the device to account for varying ambient lighting conditions despite colder weather operation of the device.

17. The electronically-operable, portable, variable, light attenuating liquid-crystal device of claim 16, wherein light transmissivity is relatively high when no electricity is produced by said second power circuit and relatively low when electricity is produced by said second power circuit.

18. The electronically-operable, portable, variable, light attenuating liquid-crystal device of claim 16, used in one of a goggle lens, a vision-screen lens and an eyeglasses lens adapted for heating to prevent fogging impairment of vision of a wearer of the lens.

19. The electronically-operable, portable, light attenuating liquid-crystal device of claim 16, used in a visual display of a wearable headset display device adapted for one of a virtual reality display and an augmented reality display and adapted for heating of the visual display to prevent fogging impairment of visibility of the display by a user of the electronic device.
